(19) United States
(12) Patent Application Publication
(10) Pub. No.: US 2016/0101071 A1
(43) Pub. Date: Apr. 14, 2016

(54) COMBINATION CANCER TREATMENT

(71) Applicant: WISCONSIN ALUMNI RESEARCH FOUNDATION, Madison, MI (US)

(72) Inventors: Vincent Leonard Cryns, Middleton, WI (US); Dmitry Malin, Madison, WI (US); Elena Strekalova, Madison, WI (US)

(21) Appl. No.: 14/692,048

(22) Filed: Apr. 21, 2015

Related U.S. Application Data
(60) Provisional application No. 61/985,095, filed on Apr. 28, 2014.

(51) Int. Cl. A61K 31/155; A61K 38/17; A61K 39/395; A61K 9/00

(52) U.S. Cl. CPC A61K 31/155; A61K 9/0053; A61K 9/0019; A61K 38/1793; A61K 39/39558

(57) ABSTRACT

Described herein is TRAIL receptor targeting therapy in combination with metformin for treatment of cancer in humans. Using TRAIL receptor targeting therapy such as the TRAIL molecule, agonistic human monoclonal antibodies against TRAIL receptors, or peptides targeting TRAIL receptors in combination with metformin for the treatment of all types of cancer allows to obtain an optimum therapeutical effect at any time of the progression of the disease.

### CROSS-REFERENCE TO RELATED APPLICATIONS

This application claims priority to U.S. Provisional Application 61/985,095 filed on Apr. 28, 2014, which is incorporated herein by reference in its entirety.

### FIELD OF THE DISCLOSURE

The present disclosure is related to the combination of TRAIL receptor agonists, such as TRAIL or monoclonal antibodies that activate proapoptotic TRAIL receptors, and a second chemotherapeutic agent for the treatment of cancer.

### BACKGROUND

Given the inherent toxicity of many chemotherapy drugs used to treat cancer, there is great interest in identifying agents that selectively target cell death pathways in cancer cells to activate a genetically programmed cellular suicide response known as “apoptosis”. One particularly promising molecular target is the tumor necrosis factor (TNF)-related apoptosis-inducing ligand (TRAIL) receptor pathway. TRAIL is a proapoptotic cytokine that plays a critical role in immune surveillance of tumors. TRAIL activates apoptosis by binding to its death receptors DR4/TRAIL-R1 and DR5/TRAIL-R2, triggering a series of protein interactions that culminate in the sequential activation of members of the caspase family of cell death proteases. Results from mice deficient in the TRAIL receptor point to a key role for TRAIL in suppressing metastases.

The TRAIL receptor pathway has emerged as a promising therapeutic target for cancer. Soluble recombinant TRAIL and agonistic antibodies targeting DR4/TRAIL-R1 and DR5/TRAIL-R2 have been shown to preferentially induce apoptosis in cancer cells and to have little effect on untransformed cells. TRAIL receptor agonists have been demonstrated to inhibit primary and metastatic tumor growth in many murine models. It has recently been demonstrated that metastatic breast cancer cells were more sensitive to apoptosis induction by a human agonistic monoclonal antibody targeting DR5/TRAIL-R2 (lexatumumab) than a DR4/TRAIL-R1 agonistic antibody (mapatumumab). Several TRAIL receptor agonists, including recombinant TRAIL (dulanermin) and a variety of humanized agonistic monoclonal antibodies targeting DR4/TRAIL-R1 or DR5/TRAIL-R2, are in clinical trials in diverse cancer types. Phase I trials have supported the safety and tolerability of these agents.

Despite the considerable promise of TRAIL receptor agonists as cancer therapies, de novo and acquired resistance has been observed and represent a major barrier to their clinical translation. Phase 2 trials of these agents alone or in combination with chemotherapy have been largely disappointing. As such, the identification of agents to prevent or reverse TRAIL-resistance would greatly enhance the therapeutic impact of TRAIL receptor agonists. Although several potential TRAIL-sensitizing agents have been identified, including aspirin, resveratrol, thiazolidinediones, histone deacetylase inhibitors and others, many of these agents have significant toxicity, poorly characterized pharmacokinetics and/or unclear long-term safety. Therefore, it is very crucial to find ways to overcome resistance to TRAIL therapy.

### BRIEF SUMMARY

In one aspect, a method of treating cancer in a human individual in need of treatment for cancer comprises co-administering metformin and a TRAIL receptor agonist, wherein the metformin is administered in an effective amount to sensitize cancer cells to the TRAIL receptor agonist.

In another aspect, a method of improving the sensitivity of a human cancer patient to TRAIL receptor agonist therapy comprises identifying a human cancer patient as a candidate for TRAIL receptor agonist therapy, and administering to the patient both metformin and a TRAIL receptor agonist, wherein metformin is administered to the patient before, during, or after administration of the TRAIL receptor agonist, and wherein the metformin is administered in an effective amount to sensitize cancer cells to the TRAIL receptor agonist.

### BRIEF DESCRIPTION OF THE DRAWINGS

FIG. 1 shows that metformin sensitizes cancer cells to TRAIL and lexatumumab. Human GILM2, HT29 and DU145 carcinoma cells were untreated or preincubated with metformin (MF, 5 mM) for 48 hours and then treated with TRAIL (1.5 μg/ml), mapatumumab (1.5 μg/ml), or lexatumumab (1.5 μg/ml) for an additional 24 hours. Cell viability was determined by MTS assay and expressed as fold-change relative to untreated controls. *P<0.05, **P<0.01 or ***P<0.001 versus controls.

FIG. 2 shows that transformed breast epithelial cells are more sensitive to the combination of metformin and TRAIL or metformin and lexatumumab than untransformed cells. Untransformed MCF-10A-Vector and transformed MCF-10A-RasV12 cells were untreated or preincubated with metformin (MF, 5 mM) for 48 hours and then treated with TRAIL (1.5 μg/ml), mapatumumab (1.5 μg/ml), or lexatumumab (1.5 μg/ml) for an additional 24 hours. Cell viability was determined by MTS assay and expressed as fold-change relative to untreated controls. **P<0.01 or ***P<0.001 versus controls.

FIGS. 3-5 show that metformin enhances the cytotoxicity of TRAIL receptor agonists against cancer cells. Crystal violet cell survival assay of GILM2 (FIG. 3), HT29 (FIG. 4) and DU145 (FIG. 5) cancer cells preincubated with or without metformin (MF, 5 mM) for 48 hours and then treated with TRAIL (1.5 μg/ml), lexatumumab (1.5 μg/ml), or mapatumumab (1.5 μg/ml) for 24 hours (GILM2, HT29) or 48 hours (DU145). Top panel: representative images. Bottom panel: quantification performed by scoring cell confluence in 3 fields in each well. *P<0.05 or ***P<0.001 versus controls.

FIGS. 6 and 7 show that transformed breast epithelial cells are more sensitive to the combination of metformin and TRAIL receptor agonists than untransformed cells. Crystal violet cell survival assay of untransformed MCF-10A-Vector cells (FIG. 6) or transformed MCF-10A-RasV12 cells (FIG. 7) preincubated with or without metformin (5 mM) for 48 hours and then treated with treated with TRAIL (1.5 μg/ml), lexatumumab (1.5 μg/ml) or mapatumumab (1.5 μg/ml) for an additional 24 hours. Top panel: representative images. Bottom panel: quantification performed by scoring cell confluence in 3 fields in each well. *P<0.05, **P<0.01 or ***P<0.001 versus controls.

FIGS. 8 and 9 show that metformin enhances caspase activation by TRAIL in cancer cells Immunoblots of GILM2, HT29 and DU145 carcinoma cells (FIG. 8) or MCF-10A-Vector and MCF-10A-RasV12 cells (FIG. 9) preincubated with or without metformin (5 mM) for 48 hours and then treated with vehicle or TRAIL (1.5 μg/ml) for an additional 16 hours.

FIG. 10 shows that metformin does not significantly increase TRAIL receptor mRNA levels in cancer cells. GILM2 breast cancer cells were cultured in control media or media containing 5 mM metformin (MF) for 72 hours, and total RNA was then isolated. TRAIL-R2 and TRAIL-R1 mRNA levels were measured by real-time PCR and normalized to expression in GILM2 cells grown in control media.

FIG. 11 shows that metformin does not significantly increase cell surface expression of TRAIL-R1 and TRAIL-R2 in cancer cells. GILM2 cells were grown in control media or media containing 5 mM metformin for 72 hours, incubated with control IgG, TRAIL-R1 or TRAIL-R2 mAb, and analyzed by flow cytometry. Grey bar: negative control. Blue line: cells cultured in control media and incubated with TRAIL-R1 or TRAIL-R2 Ab. Red line: cells grown in media with 5 mM metformin and incubated with TRAIL-R1 or TRAIL-R2 Ab.

FIG. 12 shows that metformin reduces XIAP levels in cancer cells. Immunoblot of XIAP expression in MDA-MB-231, GILM2 and MDA-MB-468 breast cancer cell lines grown in control media (V) or media containing 5 mM metformin (MF) for 72 hours.

FIG. 13 shows that silencing XIAP reduces XIAP protein levels. MDA-MB-231 cells were transfected with a scrambled siRNA (si-Control) or one of two different siRNAs targeting XIAP (si-1 XIAP or si-2 XIAP). XIAP protein levels were determined by immunoblotting 48 hours after siRNA transfection.

FIG. 14 shows that silencing XIAP sensitizes cancer cells to TRAIL. Crystal violet cell survival assay of MDA-MB-231 cells transfected with control or XIAP siRNAs and treated with vehicle or TRAIL (1 μg/ml) for 72 hours. Top: representative images. Bottom: quantification performed by scoring cell confluence in 3 fields of each well (mean ±SEM, n=3). ***P<0.001 versus the indicated comparisons.

FIG. 15 shows that silencing XIAP sensitizes cancer cells to TRAIL-induced caspase activation. MDA-MB-231 cells were transfected with control or XIAP siRNAs and treated overnight with vehicle or TRAIL (1 μg/ml). PARP, cleaved PARP and procaspase-3 were detected by immunoblotting.

FIG. 16 shows metformin enhances the antitumor effects of TRAIL in an orthotopic model of metastatic triple-negative breast cancer. GILM2-mCherry cells (1×10⁶) were injected intraductally into both 4th mammary glands of female NOD scid IL2 receptor γ chain knockout (NSG) mice. Mice were randomized into 4 groups (10 mice per group): vehicle, TRAIL alone (10 mg/kg i.p. daily for two weeks), metformin alone (250 mg/mL in drinking water), or the combination of these doses of metformin and TRAIL. Mice were treated with metformin beginning three weeks after tumor inoculation and throughout the study; TRAIL treatment was initiated 3.5 weeks after tumor cell inoculation. Left, percent original mammary tumor volume (at three weeks) in each treatment group (mean±SEM, n=10 mice per group). **P<0.01 or ***P<0.001 versus vehicle-treated mice or the indicated comparison. Right, The percentage of the surface area occupied by lung metastases. ***P<0.001 versus vehicle-treated mice.

The above-described and other features will be appreciated and understood by those skilled in the art from the following detailed description, drawings, and appended claims.

### DETAILED DESCRIPTION

It has been found that coadministration of metformin with a TRAIL receptor agonist sensitizes cells to TRAIL receptor agonist therapy and provides improved anti-cancer activity. Importantly, cancer cells that are resistant to TRAIL receptor agonist therapy have a high level of sensitivity when the TRAIL receptor agonist is coadministered with metformin.

Inhibition of the mammalian target of rapamycin (mTOR) kinase has been reported to enhance the sensitivity of cancer cells to TRAIL receptor agonists. Without being held to theory, because the diabetes drug metformin activates the nutrient sensor AMP-activated kinase (AMPK), which inhibits mTOR activity through its actions on TSC2, it was hypothesized that metformin would act synergistically with TRAIL receptor agonists to activate apoptosis in cancer cells, including cancer cells that are resistant to TRAIL receptor agonists. Metformin has emerged as a promising cancer therapy in its own right due to its systemic insulin-sensitizing effects and direct actions on tumor cells, leading to many clinical trials in diverse cancers. From a translational perspective, metformin is a particularly attractive cancer therapy because its safety has been well established over decades in many diabetic patients worldwide. As such, there would be few barriers to its clinical implementation as a cancer therapeutic in combination with TRAIL receptor agonists.

In one aspect, a method of treating cancer in a human individual in need of treatment for cancer comprises co-administering metformin and a TRAIL receptor agonist, wherein the metformin is administered in an effective amount to sensitize cancer cells to the TRAIL receptor agonist. As used herein, the term co-administering means that the two drugs are administered such that the pharmacokinetic effects of both drugs are present in the individual at the same time. The metformin and the TRAIL receptor agonist need not be administered by the same administration method, for example, metformin may be orally administered and the TRAIL receptor agonist may be intravenously administered. In addition, the metformin and the TRAIL receptor agonist need not be administered on the same schedule. For example, metformin may be administered daily, for example as an oral tablet or capsule, while the TRAIL receptor agonist may be administered daily (TRAIL) or once every two to three weeks (anti-TRAIL receptor antibodies) as an intravenous injection.

Metformin is N,N-dimethylimidodicarbonimidic diamide

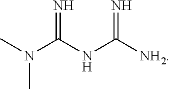

Metformin is commercially available as GLUCOPHAGE® from Bristol Myers Squibb and is also available in generic form.

In an aspect, the TRAIL agonist is a polypeptide. Dulanermin (Apo2L/TRAIL), for example, is recombinant human TRAIL which targets both TRAIL-R1 and TRAIL-R2, described for example in International Publication No. WO 2009/140469, incorporated herein by reference for its disclosure of TRAIL receptor agonists. TRAIL peptides can also be genetically linked to single chain variable fragments (scFv). The scFv can be directed against a cancer- specific target such as EGFR, anti-CD33 scFv which is directed against AML cells, or anti-CD7 scFv which is directed against acute leukemic T-cells.

In one aspect, the TRAIL agonist is an agonistic antibody that binds the TRAIL-R1/DR4 or the TRAIL-R2-DR5 receptor. Agonistic antibodies bind to and activate the receptor, mimicking the natural ligand, TRAIL. Agonistic antibodies that bind TRAIL-R1/DR4 include mapatumumab (HGS-ETR1), a fully humanized monoclonal antibody. Agonistic antibodies that bind TRAIL-R2/DR5 include lexatumumab (HGS-RTR2), drozitumab (Apomab/PRO95780), conatumumab (AMG 655), tigatuzumab (CS-1008/TRA-8), HGSTR2J/KMTRS and LBY-135. Lexatumumab, drozitumab, and conatumumab are fully human IgG1 antibodies, while tigatuzumab is a humanized IgG1 antibody and LBY135 is a chimeric mouse/human antibody. TRAIL receptors and antibodies that bind to TRAIL receptors are described in U.S. Pat. Nos. 6,455,040 (DRS); 6,743,625 (DR5); 6,433,147 (DR4), 6,902,910 (DR4); all incorporated herein by reference for their disclosure of TRAIL receptors and antibodies.

In an aspect, the TRAIL agonist is a nanobody construct as described in U.S. Publication No. 2011/0318366, incorporated herein by reference for its teaching of nanobodies. The development of nanobodies was based on the observation that the antibodies of camelidae species have functional antibodies that lack light chains, referred to as heavy-chain antibodies. Nanobodies contain a single variable antibody domain (VHH) which is stable and maintains the antigen-binding capability of the original heavy chain antibody. Nanobody technology has been developed by Ablynx®. Nanobodies can be fully humanized. A specific anti-DR5 nanobody is TAS266.

In one aspect, the metformin is orally administered and the TRAIL agonist is intravenously administered. The metformin is orally administered one to three times daily to provide a daily dosage amount of 1000 to 2550 mg per day. The TRAIL agonistic antibody is intravenously administered every two to three weeks. Lexatumumab, for example, can be administered at 0.1-10 mg/kg once every two weeks, repeated for four or more cycles of treatment. Dulanermin, for example, is administered at 8 mg/kg for 5 days, on a three week cycle.

The combination of metformin and a TRAIL receptor agonist can be used to treat any cancer, particularly breast cancer, prostate cancer, melanoma, head and neck cancer and colon cancer. In one aspect, the cancer is resistant to TRAIL receptor agonists. In another aspect, the cancer is at an advanced stage or metastatic.

In another aspect, a method of improving the sensitivity of a human cancer patient to TRAIL receptor agonist therapy comprises identifying a human cancer patient as a candidate for TRAIL receptor agonist therapy, and administering to the patient both metformin and a TRAIL receptor agonist, wherein metformin is administered to the patient before, during, or after administration of the TRAIL receptor agonist, and wherein the metformin is administered in an effective amount to sensitize cancer cells to the TRAIL receptor agonist.

There is an urgent need for new treatments for patients with solid tumors who have failed standard chemotherapy and develop metastatic disease. These patients have a poor prognosis and few treatment options. This is one clinical setting for the combination therapy of metformin and TRAIL receptor agonists. Thus, in an aspect, a candidate for TRAIL receptor agonist therapy is a patient with a solid tumor.

An “isolated” or “purified” polypeptide or fragment thereof is substantially free of cellular material or other contaminating polypeptide from the cell or tissue source from which the protein is derived, or substantially free of chemical precursors or other chemicals when chemically synthesized. The language “substantially free of cellular material” includes preparations of polypeptide in which the polypeptide is separated from cellular components of the cells from which it is isolated or recombinantly produced. Thus, polypeptide that is substantially free of cellular material includes preparations of polypeptide having less than about 30%, 20%, 10%, or 5% (by dry weight) of heterologous polypeptide (also referred to herein as a “contaminating polypeptide”). Preferably, the preparation is at least about 75% by weight pure, more preferably at least about 90% by weight pure, and most preferably at least about 95% by weight pure. A substantially pure TRAIL polypeptide may be obtained, for example, by extraction from a natural source (e.g., an insect cell); by expression of a recombinant nucleic acid encoding a TRAIL polypeptide; or by chemically synthesizing the polypeptide. Purity can be measured by any appropriate method, e.g., by column chromatography, polyacrylamide gel electrophoresis, or by high pressure liquid chromatography (HPLC) analysis.

The present disclosure also includes isolated (i.e., removed from their natural milieu) antibodies that selectively bind a TRAIL receptor or a mimetope thereof, particularly R1/DR4 and R2/DRS. As used herein, the term “selectively binds to” refers to the ability of antibodies to preferentially bind to TRAIL receptors and mimetopes thereof Binding can be measured using a variety of methods standard in the art including enzyme immunoassays (e.g., enzyme linked immunoassays (ELISA)), immunoblot assays, and the like; see, Sambrook et al., Eds., Molecular Cloning: A Laboratory Manual, 2nd ed., Cold Spring Harbor Laboratory Press, 1989, or Harlow and Lane, Eds., Using Antibodies, Cold Spring Harbor Laboratory Press, 1999.

Isolated antibodies include antibodies in serum, or antibodies that have been purified to varying degrees. Such antibodies include polyclonal antibodies, monoclonal antibodies, humanized or chimeric antibodies, anti-idiotypic antibodies, single chain antibodies, Fab fragments, fragments produced from a Fab expression library, epitope- binding fragments of the above, and the like.

Antibodies that bind to TRAIL receptors can be prepared from the intact polypeptide or fragments containing peptides of interest as the immunizing agent. The preparation of polyclonal antibodies is well known in the molecular biology art; see, e.g., Production of Polyclonal Antisera in Immunochemical Processes (Manson, ed.), (Humana Press 1992) and Coligan et al., Production of Polyclonal Antisera in Rabbits, Rats, Mice and Hamsters in Current Protocols in Immunology (1992). A host for preparation and/or administration of an antibody can mean a human or a vertebrate animal, including, but not limited to, dog, cat, horse, sheep, pig, goat, chicken, monkey, rat, mouse, rabbit, guinea pig, and the like.

A monoclonal antibody composition can be antibodies produced by clones of a single cell called a hybridoma that secretes or otherwise produces one kind of antibody molecule. Hybridoma cells can be formed by fusing an antibody-producing cell and a myeloma cell or other self-perpetuating cell line. Although numerous variations have been described for producing hybridoma cells, a method for the preparation of monoclonal antibodies is described by Kohler and Milstein, Nature 256, 495-497 (1975).

Briefly, monoclonal antibodies can be obtained by injecting mammals such as mice or rabbits with a composition comprising an antigen, thereby inducing in the animal antibodies having specificity for the antigen. A suspension of antibody-producing cells is then prepared (e.g., by removing the spleen and separating individual spleen cells by methods known in the art). The antibody-producing cells are treated with a transforming agent capable of producing a transformed or “immortalized” cell line. Transforming agents are known in the art and include such agents as DNA viruses (e.g., Epstein Bar Virus, SV40), RNA viruses (e.g., Moloney Murine Leukemia Virus, Rous Sarcoma Virus), myeloma cells (e.g., P3X63-Ag8.653, Sp2/0-Ag14), and the like. Treatment with the transforming agent can result in production of a hybridoma by means of fusing the suspended spleen cells with, for example, mouse myeloma cells. The transformed cells are then cloned, preferably to monoclonality. The cloning is preferably performed in a medium that will support transformed cells, and not support non-transformed cells. The tissue culture medium of the cloned hybridoma is then assayed to detect the presence of secreted antibody molecules by antibody screening methods known in the art. The desired clonal cell lines are then selected.

A therapeutically useful anti-TRAIL receptor antibody may be derived from a “humanized” monoclonal antibody. Humanized monoclonal antibodies are produced by transferring mouse complementarity determining regions from heavy and light variable chains of the mouse immunoglobulin into a human variable domain, then substituting human residues into the framework regions of the murine counterparts. The use of antibody components derived from humanized monoclonal antibodies obviates potential problems associated with immunogenicity of murine constant regions. Techniques for producing humanized monoclonal antibodies can be found in Jones et al., Nature 321: 522, (1986) and Singer et al., J. Immunol. 150: 2844, (1993). The antibodies can also be derived from human antibody fragments isolated from a combinatorial immunoglobulin library; see, for example, Barbas et al., Methods: A Companion to Methods in Enzymology 2, 119, (1991).

In addition, chimeric antibodies can be obtained by splicing the genes from a mouse antibody molecule with appropriate antigen specificity together with genes from a human antibody molecule of appropriate biological specificity; see, for example, Takeda et al., Nature 314: 544-546, 1985. A chimeric antibody is one in which different portions are derived from different animal species.

Anti-idiotype technology can be used to produce monoclonal antibodies that mimic an epitope. An anti-idiotypic monoclonal antibody made to a first monoclonal antibody will have a binding domain in the hypervariable region that is the “image” of the epitope bound by the first monoclonal antibody. Alternatively, techniques used to produce single chain antibodies can be used to produce single chain antibodies against TRAIL receptors, as described, for example, in U.S. Pat. No. 4,946,778. Single chain antibodies are formed by linking the heavy and light chain fragments of the FIT region via an amino acid bridge, resulting in a single chain polypeptide.

Antibody fragments that recognize specific epitopes can be generated by techniques well known in the art. Such fragments include Fab fragments produced by proteolytic digestion, and Fab fragments generated by reducing disulfide bridges.

In another method, anti-TRAIL receptor antibodies can be produced recombinantly using techniques known in the art. Recombinant DNA methods for producing antibodies include isolating, manipulating, and expressing the nucleic acid that codes for all or part of an immunoglobulin variable region including both the portion of the variable region comprised by the variable region of the immunoglobulin light chain and the portion of the variable region comprised by the variable region of the immunoglobulin heavy chain. Methods for isolating, manipulating and expressing the variable region coding nucleic acid in eukaryotic and prokaryotic hosts are disclosed in U.S. Pat. No. 4,714,681; Sorge et al., Mol. Cell. Biol. 4, 1730-1737 (1984); Beher et al., Science 240, 1041-1043 (1988); Skerra et al., Science 240, 1030-1041 (1988); and Orlandi et al., Proc. Natl. Acad. Sci. U.S.A. 86, 3833-3837 (1989).

A preferred method to produce anti-TRAIL receptor antibodies includes (a) administering to an animal an effective amount of TRAIL receptor (ranging in size from a polypeptide fragment to a full-length protein) or mimetope thereof to produce the antibodies and (b) recovering the antibodies.

Antibodies can be recovered and/or purified by methods known in the art. Suitable methods for antibody purification include purification on Protein A or Protein G beads, protein chromatography methods (e.g., diethyl-amino-ethyl (DEAE) ion exchange chromatography, ammonium sulfate precipitation), antigen affinity chromatography, and the like.

As used herein “anti-TRAIL receptor antibody” refers to an antibody capable of complexing with TRAIL receptors.

Also included herein are pharmaceutical compositions containing metformin and/or TRAIL receptor agonists. As used herein, “pharmaceutical composition” means therapeutically effective amounts of an active compound together with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

The active ingredient, particularly the TRAIL receptor agonist, may be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anesthetic, preservative and buffering agents, can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

The phrase “effective amount,” as used herein, means an amount of an agent which is sufficient enough to significantly and positively modify symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

The amount of compound effective for any indicated condition will, of course, vary with the individual subject being treated and is ultimately at the discretion of the medical or veterinary practitioner. The factors to be considered include the condition being treated, the route of administration, the nature of the formulation, the subject's body weight, surface area, age and general condition, and the particular compound to be administered. The total daily dose may be given as a single dose, multiple doses, e.g., two to six times per day, or by intravenous infusion for a selected duration.

The invention is further illustrated by the following non-limiting examples.

### Examples

### Methods

Cell culture and reagents: Human GILM2 breast carcinoma cells were graciously provided by Dr. Janet Price, MD Anderson Cancer Center, and were grown in DMEM/F12 media with 5% fetal bovine serum (FBS), 100 IU/mL penicillin/streptomycin, and 1× insulin/transferrin/sodium selenite mix (Invitrogen™). Human HT29 colon adenocarcinoma and DU145 prostate cancer cells were maintained in RPMI media supplemented with 10% FBS and 100 IU/mL penicillin/streptomycin (Invitrogen™). Human MCF-10A breast epithelial cells stably expressing the H-RasV12 oncogene or empty vector were cultured in DMEM/F12 media with 5% horse serum (Invitrogen™), 20 ng/mL epidermal growth factor, 100 ng/mL cholera toxin, 0.5 mg/mL hydrocortisone, 10 mg/mL insulin (Sigma-Aldrich®), and 100 IU/mL penicillin/streptomycin. Lexatumumab (Lexa) and Mapatumumab (Mapa) were kindly supplied by Dr. Robin Humphreys when he worked at Human Genome Sciences. Soluble recombinant TRAIL corresponding to amino acids 95-281 of the protein was expressed in bacteria, and the His-tagged protein was purified using the QIAexpress™ Protein Purification System (QIAGEN) as known in the art.

Cell viability assay: An MTS cell viability assay was performed as known in the art. Briefly, cells were plated overnight in 96-well plates (3×10³ cells/well). Cells were then untreated or preincubated with metformin (5 mM) for 48 hours before adding lexatumumab (1.5 μg/ml), mapatumumab (1.5 μg/ml) or TRAIL (1.5 μg/ml). Cell viability was assessed 24 hours later. Cell viability was determined in triplicate wells and expressed as the fold-change relative to untreated controls.

Crystal violet cell survival assay: Cells were seeded overnight on 6-well plates (3×10⁵ cells/well). Cells were then untreated or preincubated with metformin (5 mM) for 48 hour before adding lexatumumab (1.5 μg/ml), mapatumumab (1.5 μg/ml) or TRAIL (1.5 μg/ml) for an additional 24 hours (GILM2, HT29, and MCF-10A cells) or 48 hours (DU145). Surviving cells were fixed and stained with crystal violet as described in the art. The percentage of cell confluence was determined using NIH Image J software.

Immunoblotting: Cell lysates were prepared and analyzed by immunoblotting as described in the art using primary antibodies to detect actin (Sigma-Aldrich®), PARP (PHARMAGEN) and caspase-3 (Cell Signaling).

### Example 1

### Metformin Sensitizes Cancer Cells to Cell Death Induction by Some TRAIL Receptor Agonists

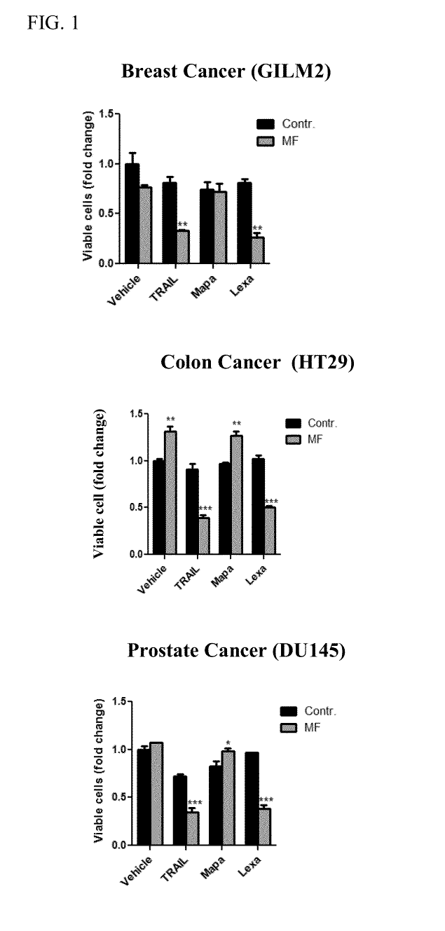

To evaluate the response of different cancer cell lines to the combination of TRAIL receptor agonists and metformin, human GILM2 breast carcinoma cells, HT29 colon cancer cells and DU145 prostate carcinoma cells were preincubated with metformin for 48 hours and then treated with lexatumumab, mapatumumab or TRAIL. Metformin enhanced the sensitivity of all three cells lines to lexatumumab and TRAIL as determined by an MTS cell viability assay (FIG. 1). In contrast, metformin did not augment the cytotoxicity of mapatumumab. Notably, metformin and TRAIL receptor agonists individually had modest or no significant effect on cell viability in these experiments.

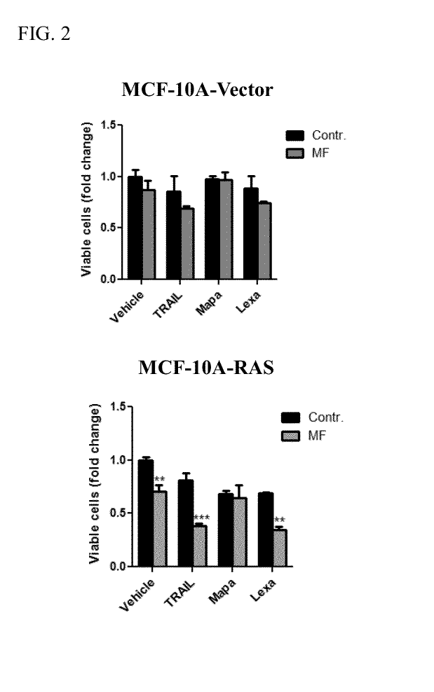

To determine whether transformed breast epithelial cells were more sensitive to the combination of metformin and TRAIL receptor agonists, MCF-10A breast epithelial cells stably expressing oncogenic H-RasV12 or empty vector were preincubated with metformin for 48 hours and then treated with lexatumumab, mapatumumab or TRAIL. Notably, metformin enhanced the sensitivity of MCF-10A-RasV12 cells to lexatumumab and TRAIL but not mapatumumab (FIG. 2), consistent with prior findings. Both metformin and TRAIL receptor agonists individually had only modest effects on the cell viability of these transformed cells. However, untransformed MCF-10A-Vector cells were largely resistant to metformin, TRAIL receptor agonists and the combination of these agents.

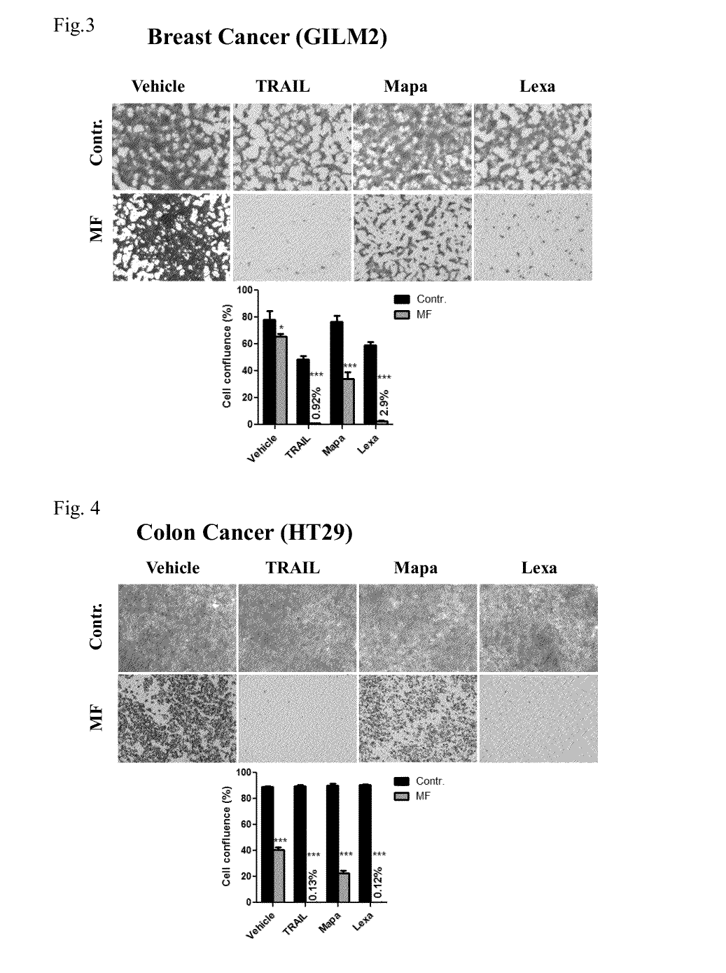

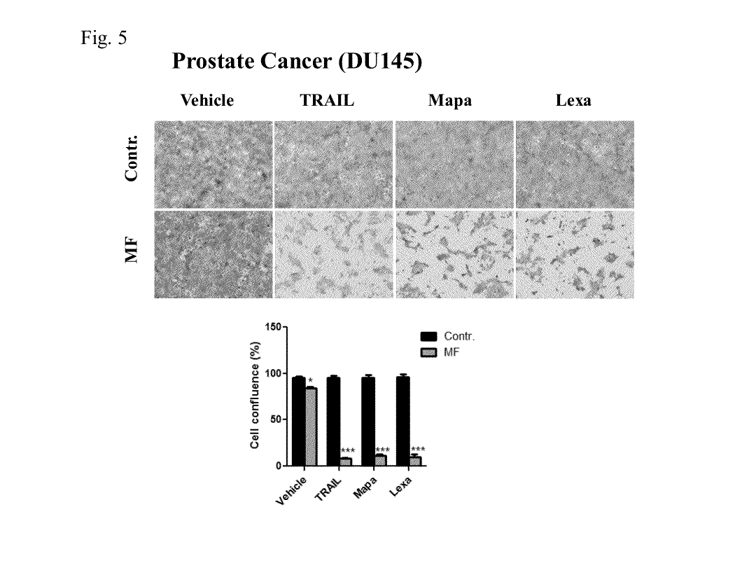

To validate these findings using a second assay, GILM2, HT29 and DU145 carcinoma cells were preincubated with metformin for 48 hours and then treated with lexatumumab, mapatumumab or TRAIL for 24-48 hours. The percentage cell confluence of surviving crystal violet-stained cells was determined using NIH Image J software. Under these conditions, metformin treatment increased the cytoxicity of TRAIL, mapatumumab and lexatumumab in all three cancer cell lines, although the effects of metformin on mapatumumab-induced cell death were more modest in GILM2 and HT29 cells (FIGS. 3-5). HT29 and DU145 cells were highly resistant to TRAIL receptor agonists alone, while TRAIL and lexatumumab induced modest cell death in GILM2 cells. Metformin alone also had modest cytoxicity against GILM2 and DU145 cells and more robust cytotoxicity against HT29 cells under these conditions.

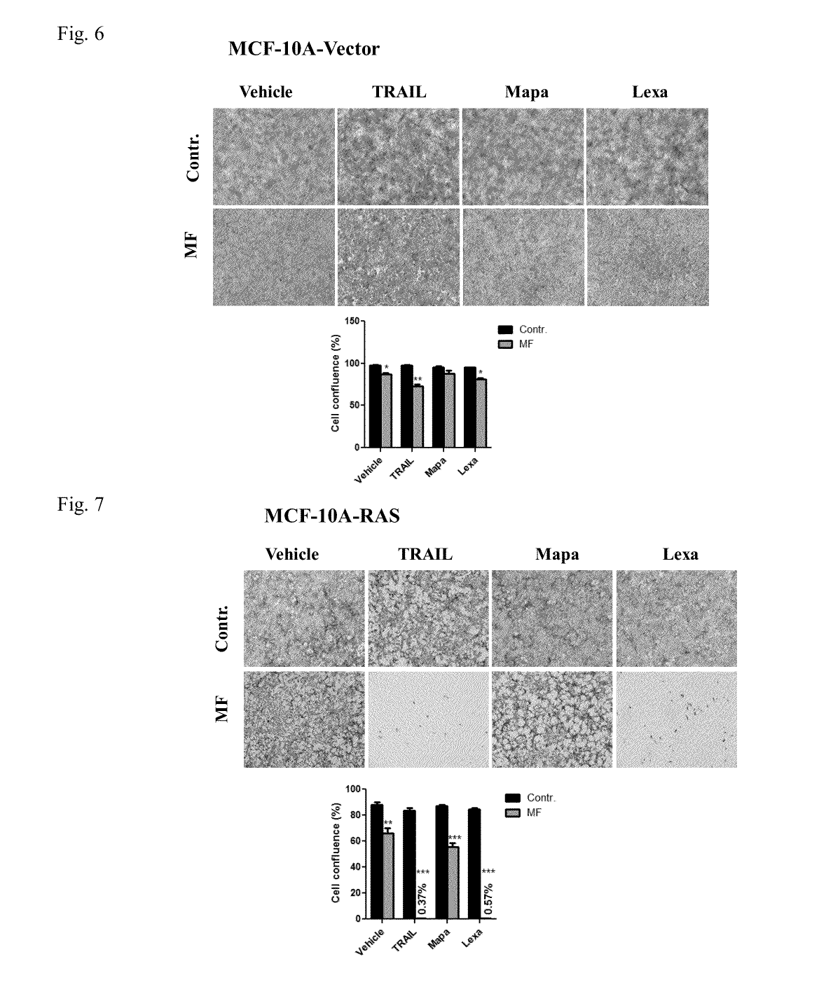

To determine the tumor-selectivity of these agents, MCF-10A-Vector and MCF-10A-RasV12 cells were preincubated with metformin and then treated with TRAIL, mapatumumab or lexatumumab. Metformin or the combination of metformin and TRAIL receptor agonists had little effect on untransformed MCF-10A-Vector cells (FIG. 6). In contrast, metformin dramatically sensitized transformed MCF-10A-RasV12 cells to lexatumumab and TRAIL, but had only a modest effect on mapatumumab-induced cell death (FIG. 7).

### Example 2

### Metformin Enhances Caspase Activation by TRAIL

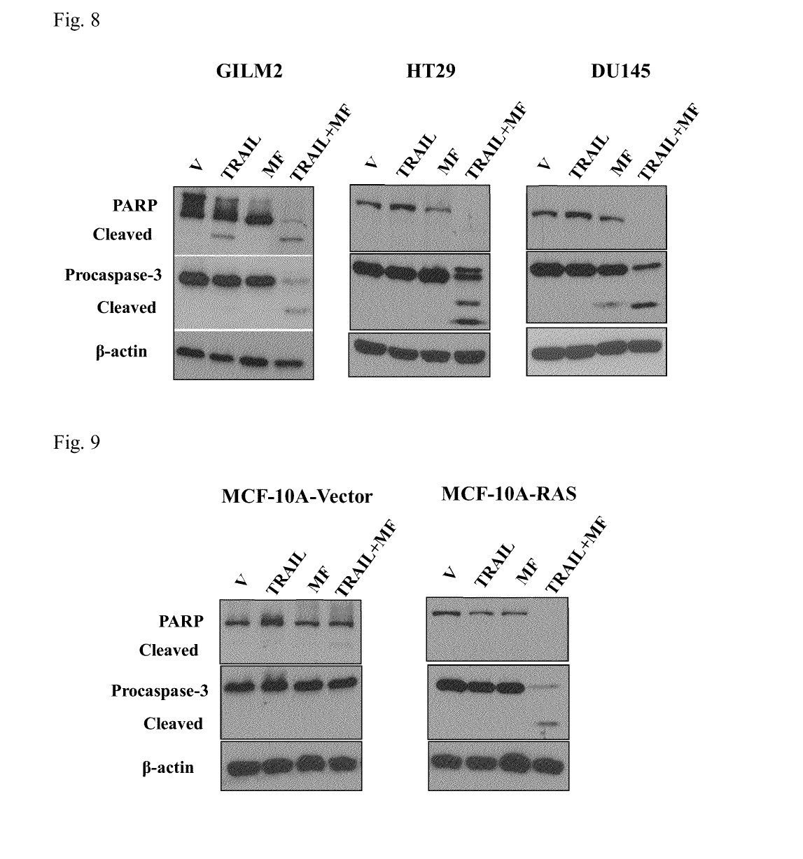

TRAIL receptor agonists initiate apoptosis by activating apical caspases-8 and caspase-10, which subsequently cleave and activate the executioner caspase-3 in the extrinsic pathway. Metformin augmented TRAIL-induced proteolytic activation of procaspase-3 to its active cleaved fragment(s) in GILM2, HT29 and DU145 carcinoma cells (FIG. 8). In addition, metformin treatment increased TRAIL-induced proteolysis of the caspase substrate PARP as detected by a reduction in the amount of full-length PARP and/or increased amount of its cleaved product. To evaluate the tumor-selectivity of these effects, MCF-10A-Vector and MCF-10A-RasV12 cells were treated with metformin, TRAIL or the combination of these agents. Consistent with the cell viability results, metformin enhanced TRAIL-induced proteolytic cleavage of procaspasase-3 and PARP in transformed MCF-10A-RasV12 cells but had little effect on untransformed MCF-10A-Vector cells (FIG. 9).

### Discussion of Examples 1 and 2

TRAIL receptor agonists have emerged as promising proapoptotic cancer therapies because of their relative tumor-selectivity in preclinical models and demonstrated safety and tolerability in phase I clinical trials. However, de novo and acquired resistance to these agents is a major barrier to their clinical translation. Here, it has been demonstrated that the diabetes medication metformin robustly sensitizes diverse cancer cell types to TRAIL receptor agonists, particularly lexatumumab (which activates DR5/TRAIL-R2) and TRAIL (which activates DR4/TRAIL-R1 and DR5/TRAIL-R2). Importantly, metformin alone or TRAIL receptor agonists alone had more modest or no effect on cell viability in these experiments. Taken together, these results indicate that metformin is a bona fide TRAIL-sensitizing agent that renders TRAIL-resistant cancer cells sensitive to TRAIL receptor agonists.

It has also been demonstrated that transformed cells are more sensitive to the combination of metformin and TRAIL receptor agonists than untransformed cells. Specifically, untransformed MCF-10A breast epithelial cells expressing an empty vector and the corresponding transformed isogenic MCF-10A cells expressing the H-RasV12 oncogene were treated with metformin, TRAIL receptor agonists or the combination. Strikingly, transformed MCF-10A-RasV12 were much more sensitive to cell death induction by the combination of metformin and TRAIL receptor agonists than the corresponding untransformed MCF-10A-Vector cells. These findings indicate that cancer cells are more susceptible to the combination of metformin and TRAIL receptor agonists than untransformed cells. The observed relative tumor-selectivity of this combination is a major therapeutic advantage over conventional chemotherapy, which has a much narrower therapeutic index and results in significant toxicity due to its actions on normal cells.

In addition, it has been shown that metformin enhances caspase activation by TRAIL in cancer cells, indicating that metformin augments the proapoptotic activity of TRAIL, even in cancer cells that are resistant to TRAIL alone. Consistent with the cell death data, treatment of untransformed MCF-10A breast epithelial cells with metformin and TRAIL receptor agonists resulted in little caspase activation, thereby underscoring the potential tumor-selectivity of this combination.

In summary, a novel combination therapy for cancer has been identified that has the potential to greatly expand the clinical impact of TRAIL receptor agonists by augmenting their proapoptotic effects and attenuating resistance. Specifically, metformin sensitizes even TRAIL-resistant cancer cells to caspase activation and cell death induction by TRAIL receptor agonists. Moreover, the combination of metformin and TRAIL receptor agonists is much more cytotoxic against cancer cells than untransformed cells, strongly suggesting that therapeutic index of this combination is likely to be high compared to conventional chemotherapy. Moreover, the well-established safety of metformin makes it a particularly attractive TRAIL-sensitizing agent with few anticipated barriers in its clinical translation.

### Example 3

### Metformin does not Increase TRAIL Receptor mRNA Levels in Cancer Cells

To determine whether metformin sensitizes cancer cells to TRAIL by increasing the expression of its proapoptotic receptors (TRAIL-R1 and TRAIL-R2), GILM2 cells were treated with metformin for 72 hours, and then TRAIL receptor mRNA levels were measured by real-time PCR. Under these conditions, metformin had little effect on TRAIL-R2 mRNA levels and modestly reduced TRAIL-R1 mRNA (FIG. 10).

### Example 4

### Metformin does not Increase Cell Surface Expression of TRAIL-R1 and TRAIL-R2 in Cancer Cells

To determine whether metformin enhances the cell surface expression of TRAIL receptors in cancer cells, GILM2 cells were treated with metformin for 72 hours, and the cell surface expression of TRAIL-R2 and TRAIL-R1 was determined by flow cytometry. Metformin did not significantly affect cell surface expression of either proapoptotic TRAIL receptor (FIG. 11). Together with the results presented in FIG. 10, these findings indicate that the TRAIL-sensitizing effects of metformin are not due to enhanced TRAIL receptor mRNA or cell surface expression in cancer cells.

### Example 5

### Metformin Reduces XIAP Levels in Cancer Cells

The antiapototic X-linked inhibitor of apoptosis protein (XIAP) has been demonstrated to confer resistance to TRAIL-induced apoptosis by suppressing caspase activation. Without being held to theory, it was postulated that metformin might sensitize cancer cells to TRAIL by downregulating XIAP. Consistent with this hypothesis, treatment of MDA-MB-231, GILM2 and MDA-MB-468 breast cancer cells with metformin for 72 hours resulted in reduction in XIAP protein levels (FIG. 12).

### Example 6

### Silencing XIAP Reduces XIAP Protein Levels

To examine the functional role of XIAP downregulation in the TRAIL-sensitizing effects of metformin, MDA-MB-231 cells were transfected with a scrambled siRNA (si-Control) or one of two different siRNAs targeting XIAP (si-1 XIAP and si-2 XIAP). Both siRNAs targeting XIAP reduced XIAP protein levels compared to the scrambled control siRNA (FIG. 13).

### Example 7

### Silencing XIAP Sensitizes Cancer Cells to TRAIL

Notably, silencing XIAP in MDA-MB-231 cells had a modest effect on cell viability and robustly sensitized these cells to TRAIL compared to a scrambled control siRNA (FIG. 14).

### Example 8

### Silencing XIAP Sensitizes Cancer Cells to TRAIL-Induced Caspase Activation

To examine whether silencing XIAP enhanced TRAIL-induced caspase activation, MDA-MB-231 cells were transfected with siRNAs targeting XIAP or a scrambled control and then treated overnight with vehicle or TRAIL. Silencing XIAP enhanced TRAIL-induced cleavage of the caspase substrate PARP (reduction of full-length PARP and/or increased cleavage product) and procaspase-3 proteolysis (reduction of procaspase-3 levels). (FIG. 15) Collectively, these results indicate that XIAP inhibits TRAIL-induced caspase activation and apoptosis, and they suggest that metformin sensitizes cancer cells to TRAIL by dowregulating XIAP.

### Example 9

### Metformin Enhances the Antitumor Effects of TRAIL in an Orthotopic Model of Metastatic Triple-Negative Breast Cancer

To determine whether metformin augments the antitumor effects of TRAIL in an ortotopic model of metastatic triple-negative breast cancer, female NSG mice bearing established GILM2-mCherry mammary tumors were treated with vehicle, metformin alone, TRAIL alone or the combination of metformin and TRAIL. (FIG. 16) Under the conditions tested, metformin had no significant effect on mammary tumor growth or lung metastases. In contrast, TRAIL inhibited mammary tumor growth, but the combination of TRAIL and metformin was more effective than TRAIL alone. Both TRAIL and metformin plus TRAIL inhibited lung metastases to a comparable degree. Collectively, these findings indicate that metformin enhances the antitumor activity of TRAIL in vivo and provide preclinical evidence supporting the combination of metformin and TRAIL or TRAIL agonists in a clinical trial

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of treating cancer in a human individual in need of treatment for cancer, comprising co-administering metformin and a TRAIL receptor agonist, wherein the metformin is administered in an effective amount to sensitize cancer cells to the TRAIL receptor agonist.

2. The method of claim 1, wherein the metformin is orally administered and the TRAIL receptor agonist is administered as an intravenous injection.

3. The method of claim 1, wherein the TRAIL receptor agonist is a polypeptide.

4. The method of claim 3, wherein the metformin is administered daily and the TRAIL receptor agonist is administered daily.

5. The method of claim 3, wherein the TRAIL receptor agonist is dulanermin.

6. The method of claim 1, wherein the TRAIL receptor agonist is an agonist antibody that binds the TRAIL-R1/DR4 or the TRAIL-R2-DR5 receptor.

7. The method of claim 6, wherein the metformin is administered daily and the TRAIL receptor agonist is administered every two to three weeks.

8. The method of claim 7, wherein the TRAIL receptor agonist is lexatumumab, mapatumumab, drozitumab, conatumumab, tigatuzumab or LBY-135.

9. The method of claim 1, wherein the TRAIL receptor agonist is a nanobody comprising a single variable antibody domain.

10. The method of claim 1, wherein the cancer is breast cancer, prostate cancer, melanoma, head and neck cancer, or colon cancer.

11. The method of claim 10, wherein the cancer is a TRAIL-resistant cancer.

12. A method of improving the sensitivity of a human cancer patient to TRAIL receptor agonist therapy, comprising
identifying a human cancer patient as a candidate for TRAIL receptor agonist therapy,
and administering to the patient both metformin and a TRAIL receptor agonist, wherein metformin is administered to the patient before, during, or after administration of the TRAIL receptor agonist, and wherein the metformin is administered in an effective amount to sensitize cancer cells to the TRAIL receptor agonist.

13. The method of claim 12, wherein the metformin is orally administered and the TRAIL receptor agonist is administered as an intravenous injection.

14. The method of claim 12, wherein the TRAIL receptor agonist is a polypeptide.

15. The method of claim 14, wherein the metformin is administered daily and the TRAIL receptor agonist is administered daily.

16. The method of claim 14, wherein the TRAIL receptor agonist is dulanermin.

17. The method of claim 12, wherein the TRAIL receptor agonist is an agonist antibody that binds the TRAIL-R1/DR4 or the TRAIL-R2-DR5 receptor.

18. The method of claim 17, wherein the metformin is administered daily and the TRAIL receptor agonist is administered once every two to three weeks.

19. The method of claim 17, wherein the TRAIL receptor agonist is lexatumumab, mapatumumab, drozitumab, conatumumab, tigatuzumab or LBY-135.

20. The method of claim 12, wherein the TRAIL receptor agonist is a nanobody comprising a single variable antibody domain.

21. The method of claim 12, wherein the cancer is breast cancer, prostate cancer, melanoma, head and neck cancer, or colon cancer.

22. The method of claim 21, wherein the cancer is a TRAIL-resistant cancer.
